(19) United States
(12) Patent Application Publication
(10) Pub. No.: US 2021/0074388 A1
(43) Pub. Date: Mar. 11, 2021

(54) Health History Updating System

(71) Applicant: Megan Lynne Morgenstern, Burbank, CA (US)

(72) Inventor: Megan Lynne Morgenstern, Burbank, CA (US)

(21) Appl. No.: 16/563,819

(22) Filed: Sep. 7, 2019

(51) Int. Cl. G16H 10/20; G16H 10/60

(52) U.S. Cl. CPC G16H 10/20; G16H 10/60

(57) ABSTRACT

A system provides interactive interfaces for users to register to the system, and matches donors of egg, sperm, or adopted child to match with, principles associated with and responsible for a child to access information from donors and professional participants.

### BACKGROUND OF THE INVENTION

### 1. Field of the Invention

The present invention is in the field of storing and disseminating medical records and pertains more particularly to records relating to parents of donor-conceived or adopted children whether the relationship is anonymous or known.

### 2. Discussion of the State of the Art

Most donors (egg, sperm, embryo) are required to complete extensive medical screenings prior to donating reproductive material, which include providing an extensive personal and family health history to such as fertility clinics, geneticists, and intended parents. This information is heavily relied on by the intended parents and fertility doctors in making a donor selection. Once a donor is selected, the donor usually agrees to make best efforts to update the intended parents or recipient of the egg, sperm, or embryo for a period of time, usually up to 21 years, with any substantial changes to their personal or family health history, as this information may affect the offspring. Even though such an agreement is made, most donors forget about the agreement years later and almost never provide the intended parents, fertility agency, reproductive attorney, or clinic with any health updates, despite there being substantial changes in their personal and family health histories. This can be very dangerous for the offspring as there may be important health information, such as new genetic carrier status information, new allergies, or other vital health information that could impact the offspring's own personal health and risk factors that will remain unknown due to the lack of disclosure. In addition, there may be health risks identified in the offspring which may be important for the donor to be advised about as these health risks may affect the donor's own personal health, her own natural children, or her other donor-conceived children.

In addition, genetic counselors rely heavily on reported ancestry of a donor when making recommendations for genetic testing and in preparing genetic counseling reports and summaries. With advancements in ancestry and DNA testing, many donors are learning that the ancestry that they self-reported during the time of their donation was not entirely accurate, but there is not a current way available to easily update the intended parents anonymously of these details, and if they donated years ago, the agency or clinic may no longer even have records on the case.

Another related limitation is that contact information for the donors and intended parents may change over time and may not be kept up to date. Therefore, getting in touch with a donor or intended parent may become increasingly more difficult as the time goes by. This may be further complicated by the fact that many clinics only keep records for a limited period of time, and donor agencies, clinics, and attorneys may go out of business. Delivering proper updates or health information in an anonymous and secure manner is almost impossible long past a time of donation. This is especially true in cases where the donor is only known by a code and is anonymous to the intended parents and vice versa.

To make matters worse, many intended parents cannot find important legal contracts or donor profiles long after donation as they may be stored in a file which has been lost or destroyed, and so they cannot even reference language related to their rights to health updates, and since the agency and clinic may not be a party to such agreements, enforcement is even more difficult.

In addition to the health update information concerns listed above, there is an additional related issue for intended parents. More and more intended parents are signing agreements with their donors which state that even though this will be an anonymous donation relationship, in the event that the donor-conceived child or children desire to meet or communicate with their donor in the future, the donor is open to this communication provided the donor-conceived children are a set age (usually either 18 or 21). While these agreements are often signed, there is no logistical solution to keep contact information updated so that an agreement may be facilitated. Many agencies and clinics will not have the same staff or anything on record about such an agreement many years later.

Therefore, what is clearly needed is a system and method for delivering personal and family health updates which solves the problems described above.

### SUMMARY OF THE INVENTION

In one embodiment of the invention a system is provided, comprising a network-connected server executing software from a non-transitory medium, providing interactive interfaces for users connecting to the network-connected server through network-connected computerized appliances, and a data repository coupled to the network-connected server which includes (a) a first interactive interface having entry fields and links enabling donors, defined as persons who have provided egg, sperm, or embryo, or have given up a child for adoption, to register to the system, registration providing identity in the system for each donor, and access to interfaces enabling the registered donor to upload information to the system, and (b) a second interactive interface having entry fields and links enabling persons who have or plan to have a child using assistance of a donor of an egg, sperm, or embryo, or who have adopted or plan to adopt a child to register to the system, registration providing identity in the system for each such person, and (c) a third interactive interface having entry fields and links enabling industry professionals currently providing or having previously provided services related to egg, sperm or embryo donation or closed adoption to register to the system, registration providing identity in the system for each industry professional, and access to interfaces enabling the registered industry professionals to upload information to the system for the verification of matches of donors and those persons who have or plan to have a child using assistance of a donor of a egg, sperm, or embryo, and (d) a matching process that organizes matched individual donors and industry professionals with individual ones of the persons who have or plan to have a child using assistance of a donor of an egg, sperm, or embryo, or who have adopted or plan to adopt a child.

Also, in one embodiment, a successful match between a donor or an industry professional with a registered person enables the registered person to anonymously access the information uploaded to the system by the donors or industry professional.

Also, in one embodiment a match is accomplished by an individual one of the registered persons who have or plan to have a child using assistance of a donor of an egg, sperm, or embryo, or who have adopted or plan to adopt a child, requesting updates from a registered donor or industry professional.

Also, in one embodiment, a match is accomplished by an industry partner matching a donor with a registered person who has or plans to have a child using assistance of a donor of an egg, sperm, or embryo, or who have adopted or plan to adopt a child. Also, in one embodiment a donor and person are matched through an anonymous code provided by one of the registered industry professionals or by a code generated by the system.

In another aspect of the invention a method is provided comprising, (a) registering donors, defined as persons who have provided egg, sperm, or embryo, or have given up a child for adoption, registration providing identity in a data repository for each donor, and access to interfaces enabling the registered donor to upload information to a data repository coupled to a network-connected server executing software from a non-transitory medium, the software providing interactive interfaces for users connecting to the network-connected server through network-connected computerized appliances, (b) registering persons who have or plan to have a child using assistance of a donor to register to the system, registration providing identity in the system for each such person, (c) registering industry professionals currently providing or having previously provided services related to egg, sperm or embryo donation or closed adoption to register to the system, registration providing access to interfaces enabling the registered industry professionals to upload information to the system, (d) executing a matching process that matches individual donors and industry professionals with individual ones of the persons who have or plan to have a child using assistance of a donor of an egg, sperm, or embryo, or who have adopted or plan to adopt a child;

In one embodiment of the method, a successful match between a donor or an industry professional with a registered person enables the registered person to anonymously access the information uploaded to the system by the donor they are matched with. Also, in one embodiment a match is accomplished by an individual one of the registered persons who have or plan to have a child using assistance of a donor of an egg, sperm, or embryo, or who have adopted or plan to adopt a child, request updates from a registered donor or industry professional.

Also, in one embodiment a match is accomplished by an industry partner matching a donor with a registered person who has or plans to have a child using assistance of a donor of an egg, sperm, or embryo, or who have adopted or plan to adopt a child.

Also, in one embodiment donor and person are matched through an anonymous code provided either by one of the registered industry professionals or by the system itself.

### BRIEF DESCRIPTION OF THE DRAWING FIGURES

FIG. 1 is a flow chart of a process in an embodiment of the invention.

FIG. 2 is a flow chart illustrating match verification in an embodiment of the invention.

### DETAILED DESCRIPTION OF THE INVENTION

A system according to an embodiment of the present invention provides a secure environment for donors (egg, sperm, and embryo) or biological parents having adopted out offspring to update the persons who have or are currently using the assistance of said donor to create offspring regarding any substantial personal or family health information or changes, genetic carrier information or changes, information or changes in available ancestry information, and any other information relative to the offspring or contemplated offspring without compromising anonymity of either party. This system also encourages intended parents and donors to keep their contact information up to date in a secure system so that communication could always be facilitated in the event of a medical emergency which warranted communication or in the event that both parties desired future communication.

This system also provides a secure place for intended parents and donors to store related contracts, donor profiles, donor photos, information provided during the time of the donation, related records, and related files for easy access in case there may be a desire to review this information in the future, legal concern, or medical emergency which required documentation.

This system also allows donors to not only answer detailed health questions about themselves and their families, but to be able to deliver such reports anonymously and securely to the parents of their donor-conceived children. Such reports might include updated health information noting any changes to their personal or family health, and also deliver a detailed family tree, any new ancestry information or genetic carrier status information, and a genetic pedigree all which can be shared easily with the children's pediatrician. A donor can also print a copy of her own report including her family tree and detailed health history to share with her own doctors and her own children's pediatrician. The system also enables an intended parent to enter their own health and family tree information and to merge it with the donor's answers, to create a full family tree and pedigree report showing both genetic parents information of any donor conceived children, which could be reviewed by a geneticist or shared with the offspring's pediatrician.

The system enables the donor to also store personal health records and related testing in one secure location so that he/she can refer back to this as needed in the future, as many clinics will not keep these for more than 8-10 years.

It will be readily understood that the systems and methods of the present invention, as generally described in the specification and illustrated in the drawings herein, may be arranged and designed in a wide variety of different configurations in addition to the specific examples provided. Thus, the following detailed description of the systems and methods, as represented in the drawings, is not intended to limit the scope defined by the appended claims but is merely representative of selected systems and methods. The following description is intended only by way of example, and simply illustrates certain concepts of the systems and methods, as disclosed and claimed herein.

The system described in embodiments herein is termed by the inventor the Donor 21 product, and is an online registry for egg donors, sperm donors, embryo donors, birth parents, parents of donor-conceived children, and intended parents to be able to register and deliver health updates both securely and anonymously to the parents of the children conceived either through third party reproduction or adoption.

There are three ways a match may be initiated. Either (1) an intended parent would register with Donor 21 and request updates from an egg donor, sperm donor, embryo donor male, embryo donor female, or birth parent, or (2) an industry partner would provide contact information for a donor, intended parent, or both so that an account may be created and facilitate a match or (3) a donor or parent would register and be connected through an anonymous code an industry partner (agency, doctor, clinic, or fertility attorney) or donor has provided.

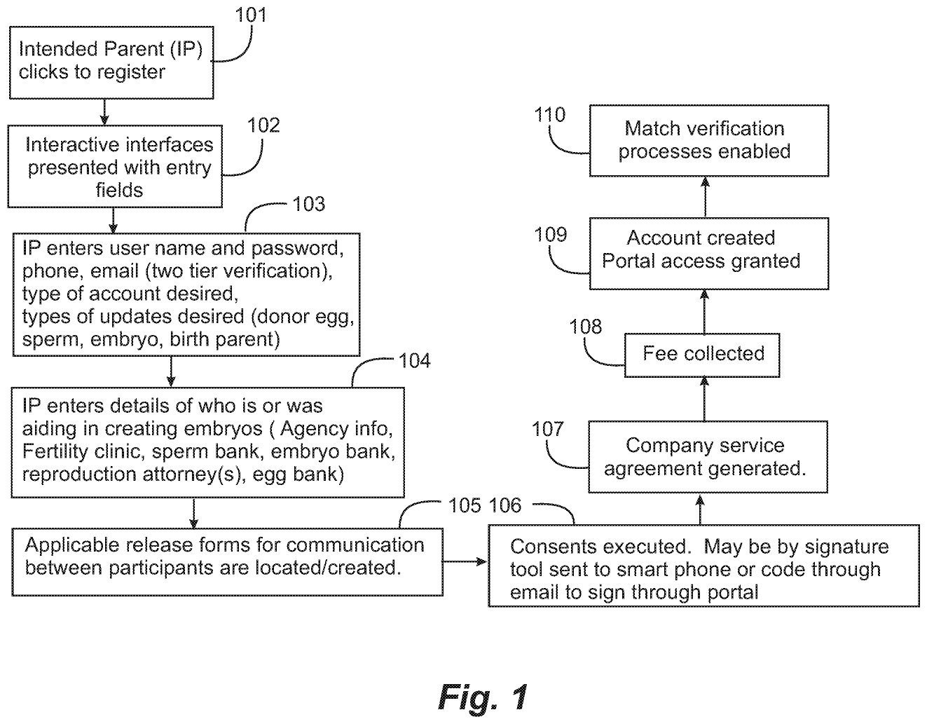

With the first method, an intended parent would go to the Donor 21 product registration and create an account. FIG. 1 is a flow chart illustrating steps in this process. At step 101 the intended parent clicks on a link provided in a page at a website hosted by the system host. At step 102 interactive interfaces are presented with entry fields. The intended parent enters, at step 103, using the entry fields and prompts provided by the system, a User Name, a Password, telephone, email (for two tier authentication), type of account desired, types of updates desired, such as donor, egg, sperm, embryo, or birth parent.

At step 104 the intended parent enters details of who is or was aiding in creating embryos, such as agency information, fertility clinic, fertility physician, donor agency, sperm bank, embryo bank, reproduction attorney, and egg bank. At step 105 applicable release forms are found or created for communication between participants. At step 106 consents are executed, either by signature tool sent to a smart phone, or over through email with a code. At step 107 a company service agreement is generated, and at step 108 a fee is collected. An account is created, and portal access is enabled at step 109. The match verification process is enabled at step 110.

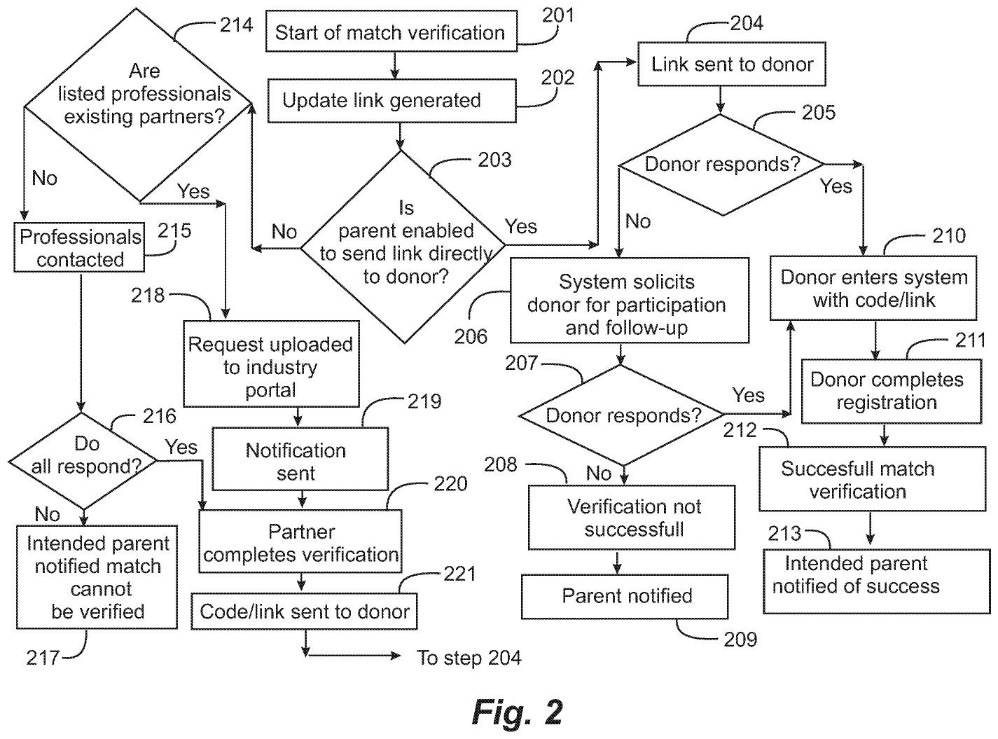

FIG. 2 is a flow chart detailing, in one embodiment, match verification, which starts at step 201. At step 202 and update link is selected by an intended parent. At step 203 the system determines whether or not the intended parent is enabled in this embodiment to send the link directly to a donor. If so, the link is sent to the donor at step 204, and at step 205 the system determines if the donor responds. If so, at step 210 the donor enters the system by the link or with a code and completes donor registration at step 211. A successful match verification occurs at step 212, and the intended parent is notified of success at step 213.

If, at step 205, the donor does not respond, the system solicits the donor at step 206 to participate. If the donor responds at step 207 control goes to step 210, and follows through steps 211 and 212, and the intended parent is notified of a successful match at step 213. If at step 207 the donor does not respond, step 208 determines that verification was not successful, and the intended parent is notified of failure at step 209.

If at step 203 the intended parent cannot send the link directly, control goes to step 214, and the system determines if there are existing professional partners. If not, then the professionals listed are contacted by the system at step 215, and at step 216 the system determines if they respond. If so, a partner completes verification at step 220, a code or link is sent to the donor at step 221, and control then goes to step 204 to follow through steps described above from step 204.

If at step 216 there is no response, then at step 217 the intended parent is notified that a match cannot be verified. If at step 214 it is determined that listed professionals are existing partners, then, at step 218 a request is uploaded to the industry portal, notification is sent at step 219, and a partner completes verification at step 220. Code/link is sent to the donor at step 221 and control goes to step 204 to follow through the steps described above following step 204.

Considering step 220, a professional partner could notify the system of a match by either a) uploading a match sheet outlining all needed match details, b) entering the match details in their secure portal, c) by calling and notifying the company at which time an agent would enter the match into the system, or d) by providing the intended parent and the donor with secure codes for registration which the system would use to identify and verify the match.

Once a match is verified the system reaches out to both the intended parent and the donor to notify them that a match was verified and to invite them to register and participate.

If the intended parent is interested, he/she/they may go through the same steps outlined above to register and create a new account, but they would enter a referral code so the system knows the account is already verified with a professional partner or a pre-registered donor. The intended parent would be notified once the donor has enrolled and agreed to participation.

With regards to the donor registering, this can also be done in a variety of ways. 1) The donor can sign up without a match in place so that she can provide medical updates to intended parents and the agency easily prior to matching. 2.) The donor can sign up upon being invited by either the system or by a professional partner he or she has worked with, such as an agency, clinic, reproductive lawyer, etc. 3.) The donor can sign up after an intended parent has registered and requested the donor to participate and the system has verified the identity of the donor.

Details of donor registration may include entry of demographics—Donor will submit identifying demographics and set up a username and password which will be protected with two-tier security, as described above for intended parents. Donor will verify her cell phone and email. Donor will identify if she/he was an egg donor, sperm donor, or embryo donor (female or male). The donor will be asked to offer information on any clinics, doctors, coordinators, banks, reproductive attorneys, and agencies he/she may have worked with and provide contact demographic information about each, as well as any donor IDs he/she may have been assigned. He/She will also be asked a few identifying questions such as eye color, race, height, and to submit a photo for confirmation of identity.

In addition, the donor will sign applicable releases to allow communication and disclosures between themselves and the intended parent and any professional partners listed above. Also, they would sign the consents with the product. Payment collected—Donor to identify where he/she wants any applicable payments/credits issued. They can also opt to not charge the intended parent for updates.

A confirmation page is displayed with access to a donor portal and intake forms. The donor is asked to complete a family tree questionnaire identifying all of her 1st and 2nd degree relatives, a lifestyle questionnaire for herself and family members related to diet, activity, smoking, and other lifestyle or behavioral choices that may impact health, a personal health questionnaire with reproductive questions and ancestry information.

After completing all sections, the donor will finalize her report and after the system approves the report, a .pdf will be uploaded to the donor portal where the donor can download a .pdf of the report to share with their pediatrician or view their genetic family tree/pedigree. If the donor is matched with a recipient and all disclosures and consents are in place, this report will be uploaded to that recipient's portal as well with any identifying information redacted. The recipient can choose to enter information on the other genetic parent of the child (whether donor sperm or donor egg or one of the parents) and create a full combined health history and genetic family tree/pedigree.

The skilled person will understand that the embodiments described above are exemplary, and that the functions may be performed in other ways within the scope of the invention. The inventor also understands that there is no guarantee of the completeness or accuracy of the information provided by donors, parents or partners, but believes that the sharing of the information that may be facilitated in embodiments of the invention will still be preferable to absence of information, which is the situation in the prior art. The scope of the invention is limited only by the claims.

What is claimed is:

1. A system, comprising:
a network-connected server executing software from a non-transitory medium, providing interactive interfaces for users connecting to the network-connected server through network-connected computerized appliances;
a data repository coupled to the network-connected server;
a first interactive interface having entry fields and links enabling donors, defined as persons who have provided egg, sperm, or embryo, or have given up a child for adoption, to register to the system, registration providing identity in the system for each donor, and access to interfaces enabling the registered donor to upload information to the system;
a second interactive interface having entry fields and links enabling persons who have or plan to have a child using assistance of a donor of an egg, sperm, or embryo, or who have adopted or plan to adopt a child to register to the system, registration providing identity in the system for each such person;
a third interactive interface having entry fields and links enabling industry professionals currently providing or having previously provided services related to egg, sperm or embryo donation or closed adoption to register to the system, registration providing identity in the system for each industry professional, and access to interfaces enabling the registered industry professionals to upload information to the system for the verification of matches of donors and those persons who have or plan to have a child using assistance of a donor of a egg, sperm, or embryo; and
a matching process that organizes matched individual donors and industry professionals with individual ones of the persons who have or plan to have a child using assistance of a donor of an egg, sperm, or embryo, or who have adopted or plan to adopt a child;
wherein a successful match between a donor or an industry professional with a registered person enables the registered person to anonymously access the information uploaded to the system by the donors or industry professional.

2. The system of claim 1 wherein a match is accomplished by an individual one of the registered persons who have or plan to have a child using assistance of a donor of an egg, sperm, or embryo, or who have adopted or plan to adopt a child, requesting updates from a registered donor or industry professional.

3. The system of claim 1 wherein a match is accomplished by an industry partner matching a donor with a registered person who has or plans to have a child using assistance of a donor of an egg, sperm, or embryo, or who have adopted or plan to adopt a child.

4. The system of claim 1 wherein donor and person are matched through an anonymous code provided by one of the registered industry professionals or by a code generated by the system.

5. A method, comprising:
registering donors, defined as persons who have provided egg, sperm, or embryo, or have given up a child for adoption, registration providing identity in a data repository for each donor, and access to interfaces enabling the registered donor to upload information to a data repository coupled to a network-connected server executing software from a non-transitory medium, the software providing interactive interfaces for users connecting to the network-connected server through network-connected computerized appliances;
registering persons who have or plan to have a child using assistance of a donor to register to the system, registration providing identity in the system for each such person;
registering industry professionals currently providing or having previously provided services related to egg, sperm or embryo donation or closed adoption to register to the system, registration providing access to interfaces enabling the registered industry professionals to upload information to the system; and
executing a matching process that matches individual donors and industry professionals with individual ones of the persons who have or plan to have a child using assistance of a donor of an egg, sperm, or embryo, or who have adopted or plan to adopt a child;
wherein a successful match between a donor or an industry professional with a registered person enables the registered person to anonymously access the information uploaded to the system by the donor they are matched with.

6. The method of claim 5 wherein a match is accomplished by an individual one of the registered persons who have ore plan to have a child using assistance of a donor of an egg, sperm, or embryo, or who have adopted or plan to adopt a child, request updates from a registered donor or industry professional.

7. The method of claim 5 wherein a match is accomplished by an industry partner matching a donor with a registered person who has or plans to have a child using assistance of a donor of an egg, sperm, or embryo, or who have adopted or plan to adopt a child.

8. The method of claim 5 wherein donor and person are matched through an anonymous code provided either by one of the registered industry professionals or by the system itself.
